(19) United States
(12) Patent Application Publication
(10) Pub. No.: US 2024/0188644 A1
(43) Pub. Date: Jun. 13, 2024

(54) TOURNIQUET SYSTEM

(71) Applicant: EXIGENT ENTERPRISES LLC, Chandler, AZ (US)

(72) Inventor: Shawn Kinsey, Chandler, AZ (US)

(73) Assignee: EXIGENT ENTERPRISES LLC, Chandler, AZ (US)

(21) Appl. No.: 18/531,615

(22) Filed: Dec. 6, 2023

Related U.S. Application Data
(60) Provisional application No. 63/430,968, filed on Dec. 7, 2022.

(51) Int. Cl. A41B 1/08

(52) U.S. Cl. CPC A41B 1/08; A61B 17/1322

(57) ABSTRACT

There is an article of clothing configured to store a tourniquet, comprising: a base clothing layer, including: a first plurality of hook and loop attachment regions arranged in a U-shape; a flap of flexible material fixedly coupled to the base clothing layer adjacent to a top end of the U-shape, and extending downwardly coextensive with the first plurality of hook and loop attachment regions, and including: a second plurality of hook and loop attachment regions positioned and configured to mate with the first plurality of hook and loop attachment regions; and a tab protruding beyond the second plurality of hook and loop attachment regions; and wherein when the flap of flexible material is fully coupled to the base clothing layer by mating of the first plurality of hook and loop attachment regions and the second plurality of hook and loop attachment regions a storage cavity is formed.

### BACKGROUND OF THE INVENTION

### Cross-Reference to Related Applications

This invention claims priority, under 35 U.S.C. § 120, to the U.S. Provisional Patent Application No. 63/430,968 to Shawn Kinsey filed on Dec. 7, 2022, which is incorporated by reference herein.

### Field of the Invention

The present invention relates to first aid, specifically tourniquets and tourniquet systems and kits for performing first aid.

### DESCRIPTION OF THE RELATED ART

In the related art, it has been known to use tourniquets to stop blood flow from traumatic injuries. A tourniquet is generally a tightly tied band applied around a body part (for example, a bandage tightened around an arm or a leg by twisting) in an attempt to arrest bleeding by forcibly compressing a blood vessel. A tourniquet is typically applied as a last resort method when bleeding cannot be controlled through alternative methods and the amount of blood loss is likely to cause death in seconds to minutes.

Animal attacks as well as trauma from exploding devices and gunshot wounds results in a high incidence of life and limb-threatening injuries. Exsanguination from peripheral vascular injury is the primary preventable cause of death in field trauma and has been well documented in literature regarding current war trauma. Blood loss from non-fatal injuries to arms and legs also can contribute to morbidity to kidneys, liver and other vital organs due to hypotension. The loss of significant amounts of blood also increases the rate of consumption of blood products from the blood bank. Multiple extremity wounds can also complicate triage.

It is axiomatic that there is a “platinum 5 minutes” for major vascular injury. Even with quick triage, it often takes hours to transport casualties to a medical center, and even if the distance is small, the hazardous nature of the injury locations frequently prevents medical personnel from quickly reaching the wounded. Hence the need for individuals to have a readily available tourniquet that is safe and effective.

Some improvements have been made in the field. Examples of references related to the present invention are described below in their own words, and the supporting teachings of each reference are incorporated by reference herein:

U.S. Pat. No. 7,981,135, issued to Thorpe, discloses the invention provides an article of clothing having a tourniquet affixed to the clothing at one end of the tourniquet, the tourniquet comprising a band comprised of an elastic and flexible material, the band having a width and a length, a first end and a second end, and a first face and second face, where at least a portion of the first face comprises hook-type fastener means, and where the second face comprises loop-type fastener means.

U.S. Pat. No. 9,730,704, issued to Rose et al., discloses a tourniquet system embedded within the garment, the tourniquet system including a band, an optional bar, and an anti-pinch plate. The bar comprises an elongate portion of material having a first end portion, an intermediate portion, and a second end portion, at least one aperture formed in the bar so as to accept the band and allow the band to pass therethrough, at least one locking protrusion that extends from at least one of the first end portion or the second end portion. The anti-pinch plate includes one or more band receiving apertures formed so as to accept the band and allow the band to pass therethrough, wherein the anti-pinch plate includes one or more locking notches formed substantially along an edge portion of the anti-pinch plate, wherein the locking notches provide a means for securing at least a portion of the bar to the anti-pinch plate.

U.S. Pat. No. 11,160,560, issued to Rose et al., discloses a tourniquet system embedded within the garment, the tourniquet system including a band, an optional bar, and an anti-pinch plate. The bar comprises an elongate portion of material having a first end portion, an intermediate portion, and a second end portion, at least one aperture formed in the bar so as to accept the band and allow the band to pass therethrough, at least one locking protrusion that extends from at least one of the first end portion or the second end portion. The anti-pinch plate includes one or more band receiving apertures formed so as to accept the band and allow the band to pass therethrough, wherein the anti-pinch plate includes one or more locking notches formed substantially along an edge portion of the anti-pinch plate, wherein the locking notches provide a means for securing at least a portion of the bar to the anti-pinch plate.

U.S. Patent Application Publication No.: 2008/0221612, by Rose, discloses an internal tourniquet system may comprise as many as eight tourniquets strategically positioned, two per extremity, so as to optimize the inversely proportional relationship between the force applied to the tourniquet and the blood flow from the laceration on the extremity farther from the body than the positioned tourniquet. Each garment is equipped with internal channels to position the tourniquets and each garment is equipped with removable covering flaps for ease of access to the tightening and securing mechanism on the tourniquets. Each garment may also be supplied with a lever or bar and a storage location on the garment in order to apply more torque if necessary to the tourniquet banded site. And each garment may also be supplied with such flaps that are able to secure the lever or bar in position, once blood flow has ceased to an acceptable level.

U.S. Patent Application Publication No.: 2021/0259332 by Nance, discloses a clothing article including a tourniquet disclosed. In one implementation, the clothing article is worn high at the waist or on the leg. The clothing article includes a belt. The belt presents a holster having a tourniquet. The tourniquet provides a tourniquet strap, a windlass rod and a V-ring. The tourniquet strap mounts to the holster and the windlass rod mounts to the tourniquet strap. When a user gets injured on the leg, the user tightens the tourniquet strap by twisting the windlass rod to further tighten the tourniquet strap such that bleeding stops from the injured area. Further, the V-ring locks the position of the windlass rod once it is determined that adequate pressure is achieved on the leg. In another implementation, the clothing article comes in a vest configuration and the tourniquet mounts at sleeves of the clothing article. The tourniquet helps to stop blood flow to the injured area on the arm.

The inventions heretofore known suffer from a number of disadvantages which include: not being easily visible; being complicated or difficult to use; not being quickly or easily accessible; and not being conveniently located.

What is needed is a tourniquet system that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available tourniquet systems. Accordingly, the present invention has been developed to provide an article of clothing configured to store a tourniquet.

In one embodiment, the article of clothing may comprise: a base clothing layer, that may include: a first plurality of hook and/or loop attachment regions that may be arranged in a U-shape, the U-shape may have an open top end and/or a closed bottom end. The article of clothing may also comprise: a flap of flexible material that may be fixedly coupled to the base clothing layer adjacent to the top end of the U-shape, and/or extending downwardly coextensive with the first plurality of hook and/or loop attachment regions, and/or may include: a second plurality of hook and/or loop attachment regions that may be positioned and/or configured to mate with the first plurality of hook and/or loop attachment regions; and/or a tab that may protrude beyond the second plurality of hook and/or loop attachment regions. Further, when the flap of flexible material is fully coupled to the base clothing layer by mating of the first plurality of hook and/or loop attachment regions and/or the second plurality of hook and loop attachment regions a storage cavity may be formed.

In another embodiment of the invention, a tourniquet may be disposed within the storage cavity. More, the tourniquet may not be fixedly coupled to the base clothing layer. Furthermore, the flap of flexible material may include a hole that may be disposed therethrough and/or may be disposed near a top end of the U-shape.

In an additional embodiment of the invention, the article of clothing may be a shirt and/or the base layer may be a sleeve of the shirt, and/or the top end of the U-shape may be disposed closer to a shoulder of the shirt than the bottom end of the U-shape. The shirt may comprised of spandex and/or nylon and/or polyester. Also, the hook and/or loop attachment regions may each include three patches: a first patch that may be positioned vertically along a side of the U-shape; a second patch that may be positioned horizontally along a base of the U-shape; and/or a third patch positioned vertically along a side of the U-shape opposite the first patch.

In yet another embodiment of the invention, there may be an article of clothing that may be configured to store a tourniquet, that may comprise: a base clothing layer, that may include: a first pull-release corrosion-proof coupling system that may be arranged in a U-shape, the U-shape may have an open top end and/or a closed bottom end. The article of clothing may also comprise: a flap of flexible material that may be fixedly coupled to the base clothing layer adjacent to the top end of the U-shape, and/or that may extend downwardly coextensive with the first pull-release corrosion-proof coupling system, and/or may include: a second pull-release corrosion-proof coupling system positioned and/or configured to mate with the first pull-release corrosion-proof coupling system; and/or a tab protruding beyond the second pull-release corrosion-proof coupling system. Additionally, when the flap of flexible material is fully coupled to the base clothing layer by mating of the first pull-release corrosion-proof coupling system and the second pull-release corrosion-proof coupling system a storage cavity may be formed.

In a further embodiment, the first pull-release corrosion-proof coupling system and/or the second pull-release corrosion-proof coupling system may comprises a plurality of plastic snaps. Too, the pull-release corrosion-proof coupling systems may each include three arrays: a first array that may be positioned vertically along a side of the U-shape; a second array that may be positioned horizontally along a base of the U-shape; and/or a third array that may be positioned vertically along a side of the U-shape opposite the first array.

Yet, in one embodiment, there may be an article of clothing configured to store a tourniquet, that may comprise: a base layer, that may be removably coupleable to the article of clothing and/or that may include a first pull-release corrosion-proof coupling system, that may be disposed along an inner surface of the base layer. The article of clothing may also comprise: a flap layer fixedly coupled to and/or disposed over the base layer opposite the article of clothing, wherein the flap layer may be a flexible material and may include: a second pull-release corrosion-proof coupling system positioned and/or configured to mate with the first pull-release corrosion-proof coupling system; and/or a tab that may protrude beyond the second pull-release corrosion-proof coupling system. When the flap layer may be fully coupled to the base layer by mating of the first pull-release corrosion-proof coupling system and/or the second pull-release corrosion-proof coupling system a storage cavity may be formed.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawing(s). It is noted that the drawings of the invention are not to scale. The drawings are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a front perspective view of a tourniquet system coupled to an article of clothing, according to one embodiment of the invention.

FIG. 2 is a front perspective view of a tourniquet system coupled to an article of clothing, showing a tourniquet disposed within a storage cavity, according to one embodiment of the invention.

FIG. 3 is a front perspective view of a tourniquet system coupled to a sleeve of an article of clothing, showing a tourniquet disposed within a storage cavity, according to one embodiment of the invention.

FIG. 4 is a front perspective view of a tourniquet system, showing a storage cavity and a tourniquet disposed within the storage cavity, according to one embodiment of the invention.

FIG. 5 is a front perspective view of a tourniquet system, showing a storage cavity and a tourniquet disposed outside of the storage cavity, according to one embodiment of the invention.

FIG. 6 is a front perspective view of a tourniquet system, showing a storage cavity and a tourniquet disposed outside of the storage cavity, according to one embodiment of the invention; and

FIG. 7 is a front perspective view of a tourniquet system, showing a storage cavity and a tourniquet disposed outside of the storage cavity, according to one embodiment of the invention.

### DETAILED DESCRIPTION OF THE INVENTION

For the purposes of promoting an understanding of the principles of the invention, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc.

Each statement of an embodiment, or example, is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps. “Comprising” is to be interpreted as including the more restrictive terms “consisting of” and “consisting essentially of.”

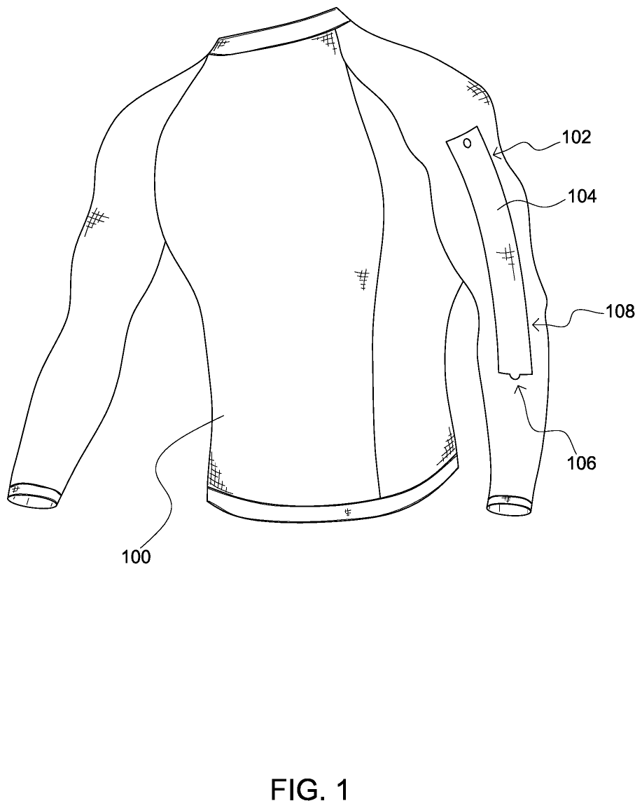

FIG. 1 illustrates a front perspective view of a tourniquet system coupled to an article of clothing, according to one embodiment of the invention. There is shown an article of clothing 100 with a base clothing layer 102. A flap of flexible material 104, including a tab 106, is shown disposed over the base clothing layer 102. When the flap of flexible material 104 is disposed over the base clothing layer 102 a storage cavity is formed therebetween 108.

The illustrated article of clothing 100 may be configured to store items within the storage cavity 108. For instance, a tourniquet may be stored within the storage cavity 108. Accordingly, the article of clothing 100 may include a storage cavity 108 of any size and/or shape for storing items. It is understood that the article of clothing 100 may be any article of clothing, such as but not limited to: a T-shirt, a long-sleeved shirt, a pair of pants, a pair of shorts, a hat, a sock, etc. In one non-limiting embodiment, the article of clothing 100 may be an athletic sleeve or a compression sleeve that is slid on and worn over a limb. For example, the article of clothing 100 may be such as a Nike Pro Elite Sleeve 2.0, made by Nike, Inc., One Bowerman Driver, Beaverton, OR, 97005.

As shown, the storage cavity 108 is formed by a base clothing layer 102 and a flap of flexible material 104. The base clothing layer 102 may be integral to the article of clothing 100 such that the base clothing layer 102 is the article of clothing 100, or the base clothing layer 102 may be separate from the article of clothing 100 such that the base clothing layer 102 is different from the article of clothing 100. For example, the base clothing layer 102 may be a separate clothing layer that couples to and/or rests on the article of clothing 100. Or the base clothing layer 102 may not be a separate layer and may be the article of clothing 100 itself. In one embodiment, the storage cavity 108 may be a pouch, and the article of clothing 100 may be a shirtsleeve.

The illustrated storage cavity 108 is formed along a sleeve of a shirt. The storage cavity 108 may be a pouch or pocket-like receptacle. In one embodiment, a tourniquet may be disposed within the storage cavity 108. The storage cavity 108 may be formed by fixedly or removably coupling the base clothing layer 102 and the flap of flexible material 104 to the shirt. The storage cavity 108 may be formed along a different portion of the shirt, such as the trunk or body of the shirt. The storage cavity 108 may also be coupled to another item or object, such as, but not limited to: a pair of pants; a jacket; a tool box; a surf board; and a first aid kit.

In one embodiment, the flap of flexible material 104 may be permanently affixed to a sleeve along an end of the flap of flexible material 104. The flap of flexible material 104 may be permanently affixed to a shirtsleeve at an end nearest a shoulder of the garment. The end of the flap of flexible material 104 nearest the shoulder may contain a grommet or hole disposed therethrough. The flap of flexible material 104 may have four sides with one side being fixed to a garment, and three sides being removable from coupling to the garment. The sides of the flap of flexible material 104 may be removably coupled to the garment by hook and loop (Velcro). In another embodiment, the flap of flexible material 104 may be rectangular and have four sides: two sides may be two inches wide, and two sides may be eight inches long.

The illustrated flap of flexible material 104 includes a tab 106 that extends downwardly from the flap of flexible material 104 beyond the base clothing layer 102. The tab 106 may be gripped for pulling on the flap of flexible material 104 and/or opening and/or accessing the storage cavity 108. The tab 106 may be any extension or device for gripping, such as but not limited to: an extension of flexible material, a D-ring, a rubber tab, a plastic tab, or a metal ring.

An end of the flap of flexible material 104, opposite the shoulder, and nearest an elbow, is illustrated to include a small, half-moon tab 106. The tab 106 may be fixed or sewn to the rip-away flap of flexible material 104. The tab 106 may be a finger-like extension of the flap of flexible material 104. In one embodiment, the tab 106 may be free-floating, not fixed to the garment, and/or resting on the garment so that a user may easily grasp the tab 106 and pull open the flap of flexible material 104 to extract a tourniquet.

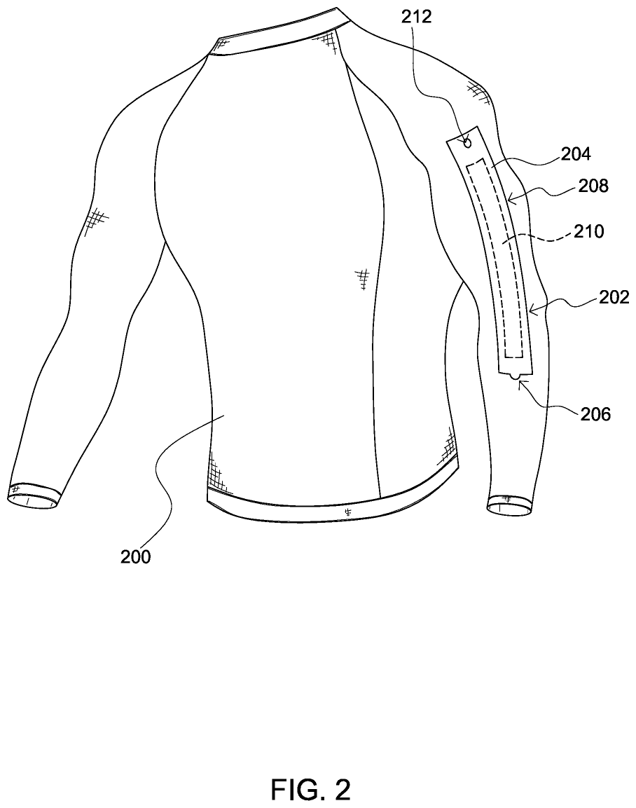

FIG. 2 illustrates a front perspective view of a tourniquet system coupled to an article of clothing, showing a tourniquet disposed within a storage cavity, according to one embodiment of the invention. There is illustrated an article of clothing 200. The article of clothing 202 includes a base clothing layer 202 with a flap of flexible material 204 disposed over the base clothing layer. The flap of flexible material 204 includes a tab 206. There is a storage cavity 208 formed by the base clothing layer 202 and the flap of flexible material 204. A tourniquet 210 is shown disposed within the storage cavity 208. The flap of flexible material 204 also includes a hole 212.

As shown, the flap of flexible material 204 includes a hole 212 disposed therethrough. The illustrated hole 212 is disposed near a top of the article of clothing 200 but may be placed anywhere along the flap of flexible material 204. In one embodiment, the hole 212 may function as a port or a drain for the storage cavity 208.

The hole 212 is disposed near an end of the flap of flexible material 204. The hole 212 may include a grommet, such as an eyelet placed through the hole 212. The hole 212 may provide the storage cavity 208 with ventilation and/or draining. For instance, the hole may allow liquids, such as salt water, to drain from the pouch.

Shown disposed at an end of the flap of flexible material 204 opposite the hole 212 is a tab 206. The tab 206 is coupled to the flap of flexible material 204 and extends therefrom. The extending tab 206 allows one to grip the tab 206 and tear open the storage cavity 208 for access to contents of the storage cavity 208. The tab 206 may be fixedly coupled to the flap of flexible material 204, or the tab 206 may be a continuous extension of the flap of flexible material 204.

A tourniquet 210 is shown folded and disposed within the cavity 208. In one embodiment, a second tourniquet may be folded, disposed, and reloaded within the cavity 208 once a first tourniquet is removed. The tourniquet 210 may be comprised of a variety of textiles and materials, such as, but not limited to, a nylon-like material or something that does not deteriorate in water, sun, salt, the elements, etc.

In one embodiment of the invention, there may be a shirt or top-half apparel 200 that houses a potentially life-saving tourniquet 210 on each one of its sleeves. The top-half apparel may be a rash guard for surfing, tactical apparel for military or law enforcement, hunting apparel or other apparel for outdoor sports, etc. The apparel 200 with tourniquet 210 may be a wearable, life-assist system, that may be used in multiple settings.

In operation of one embodiment of the invention, there may be apparel 200 that includes a life-saving tool in the form of a tourniquet 210 disposed within a cavity 208. The cavity 208 may be sealed with hook and loop so that the tourniquet may be easily accessed and removed from the cavity 208. The cavity 208 may include a flap 204 with a tab 206 that may be pulled on to undo the hook and loop seal and expose the tourniquet 210. The tourniquet 210 may be entirely removed from the cavity 208 for use. The cavity 208 may be formed on a top portion of a garment sleeve.

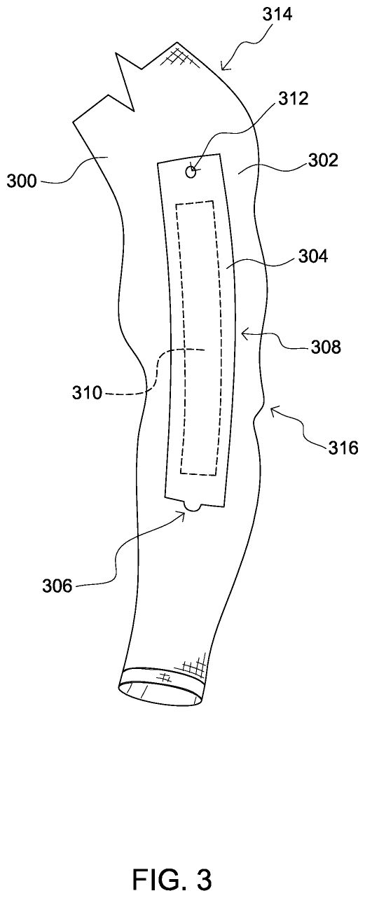

FIG. 3 illustrates a front perspective view of a tourniquet system coupled to a sleeve of an article of clothing, showing a tourniquet disposed within a storage cavity, according to one embodiment of the invention. There is shown, an article of clothing 300. The article of clothing 300 is illustrated as a sleeve 316 of a shirt 314. The article of clothing 300 includes a base clothing layer 302. A flap of flexible material 304, with a tab 306 and a hole 312, is disposed over the base clothing layer 302 and a storage cavity 308 is formed therebetween. A tourniquet 310 is disposed within the storage cavity 308.

The illustrated article of clothing 300 is a shirt 314, and the base clothing layer 302 is a sleeve 316 of the shirt 314. The shirt may be a shirt worn by a first responder, an outdoorsman, or a surfer, such as a rash guard. For example, in one embodiment the shirt 314 may be comprised of spandex and nylon. In another embodiment, the shirt 314 may be comprised of spandex and polyester.

Further, the article of clothing 300 may be a long sleeve shirt, a short sleeve shirt, a work shirt, etc. The article of clothing 300 may be a rash guard for surfing. For example, the article of clothing may be an athletic shirt made of spandex or nylon or polyester. The shirt may protect the wearer against rashes caused by abrasion, or by sunburn. Accordingly, the article of clothing may be comprised of a variety of materials, such as, but not limited to: cotton; spandex; nylon; polyester; and combinations or blends thereof.

As illustrated, the storage cavity 308 may be formed along a shirtsleeve 316. The storage cavity 308 may be a pouch with four ends or sides: a first end; a second end; a third end; and a fourth end. A tourniquet 310 is shown disposed within the storage cavity 308. The tourniquet 310 may be folded prior to disposal within the storage cavity 308.

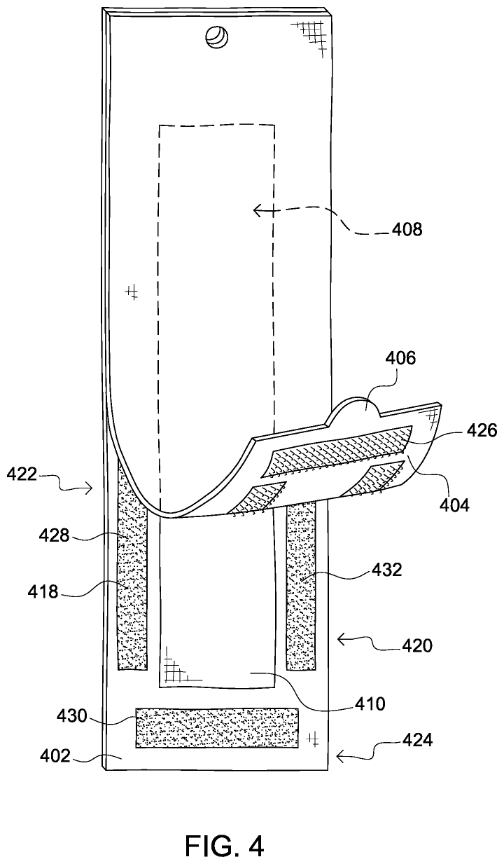

FIG. 4 illustrates a front perspective view of a tourniquet system, showing a storage cavity and a tourniquet disposed within the storage cavity, according to one embodiment of the invention. There is illustrated, a base clothing layer 402 with a flap of flexible material 404 coupled to the base clothing layer 402. The flap of flexible material 404 includes a tab 406. A storage cavity 408 is formed between the base clothing layer 402 and the flap of flexible material 404 when the flap of flexible material 404 is disposed over the base clothing layer 402. A tourniquet 410 is disposed within the storage cavity 408. The base clothing layer 402 includes a first plurality of hook and loop attachment regions 418 arranged in a U-shape 420. The U-shape 420 has an open top end 422 and a closed bottom end 424. The flap of flexible material 404 includes a second plurality of hook and loop attachment regions 426 that mate with the first plurality of hook and loop attachment regions 418. Each plurality of hook and loop attachment regions 418 and 426 has a first patch 428, a second patch 430, and a third patch 432.

As shown, the base clothing layer 402 includes a first plurality of hook and loop attachment regions 418. The first plurality of hook and loop attachment regions 418 are shown arranged in a U-shape 420 with an open top end 422 and a closed bottom end 424. However, the first plurality of hook and loop attachment regions 418 may have any arrangement for coupling to the second plurality of hook and loop attachment regions 426. For example, there may be any number of hook and loop attachment regions 418 and 426 such as one, four, or six. Likewise, the hook and loop attachment regions 418 and 426 may have any configuration or shape, such as U-shaped, C-shaped, L-shaped, chevron-shaped, and so on.

The illustrated flap of flexible material 404 is shown fixedly coupled to the base clothing layer 402 adjacent to the top end 422 of the U-shape 420. Further, the top end 422 of the U-shape 420 is disposed closer to a top of an article of clothing than the bottom end 424 of the U-shape 420. For example, if the base clothing layer 402 is a shirt, then the top end 422 of the U-shape 420 is disposed closer to a shoulder of a shirt than the bottom end 424 of the U-shape 420.

The illustrated flap of flexible material 404 also extends downwardly, coextensive with the first plurality of hook and loop attachment regions 418. The flap of flexible material 404 includes the second plurality of hook and loop attachment regions 426. The flap of flexible material 404 also includes a tab protruding beyond the second plurality of hook and loop attachment regions 426.

As shown, the second plurality of hook and loop attachment regions 426 are positioned and configured to mate with the first plurality of hook and loop attachment regions 418. Accordingly, the second plurality of hook and loop attachment regions 426 may also have a U-shape. When the flap of flexible material 404 is fully coupled to the base clothing layer 402 by mating of the first plurality of hook and loop attachment regions 418 and the second plurality of hook and loop attachment regions 426 a storage cavity is formed 408.

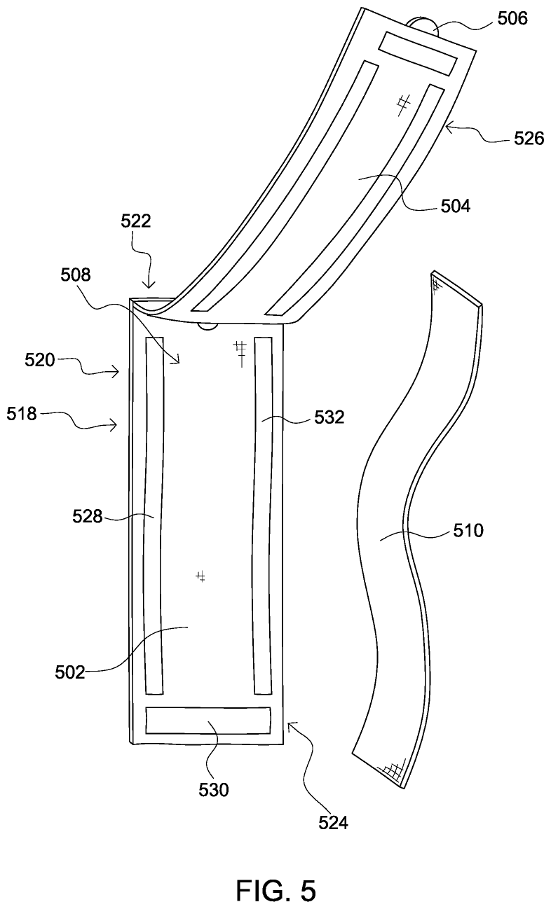

FIG. 5 illustrates a front perspective view of a tourniquet system, showing a storage cavity and a tourniquet disposed outside of the storage cavity, according to one embodiment of the invention. There is shown, a base clothing layer 502 with a flap of flexible material 504 removably and fixedly coupled to the base clothing layer 502. The flap of flexible material 504 includes a tab 506. A storage cavity 508 is formed between the base clothing layer 502 and the flap of flexible material 504 for storage of a tourniquet 510. The base clothing layer 502 includes a first pull-release corrosion-proof coupling system 518 that has a U-shape 520 with an open top end 522 and a closed bottom end 524. There is also shown a second pull-release corrosion-proof coupling system 526 that is disposed along the flap of flexible material 504 and removably couples to the first pull-release corrosion-proof coupling system 518 along a first array 528, a second array 530, and a third array 532 of each of the pull-release corrosion-proof coupling systems 518 and 526.

The illustrated tourniquet 510 is shown separate and loose from the storage cavity 508. Accordingly, the tourniquet 510 is not fixedly coupled to the base clothing layer 502. However, the tourniquet 510 may be removably coupled to the base clothing layer 502. For instance, the tourniquet 510 may be snapped to, tied to, or otherwise removably coupled to the base clothing layer 502.

As illustrated by FIGS. 4 and 5, the storage cavity 508 may be opened and closed by a pull-release corrosion-proof coupling system 518, 526. The storage cavity 508 may be opened, and the tourniquet 510 may be removed therefrom. In addition, the storage cavity 508 may be opened, a tourniquet 510 may be placed inside of the storage cavity 508, and the storage cavity 508 may be closed for storage of the tourniquet 510.

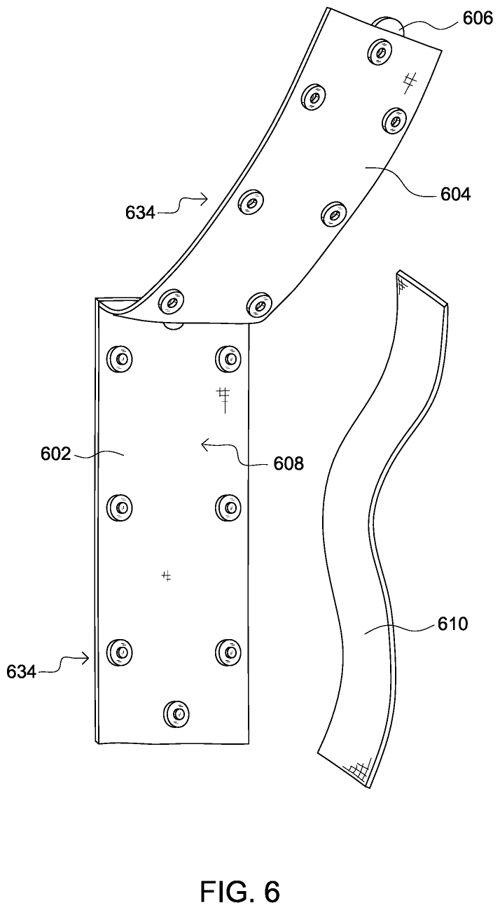

FIG. 6 illustrates a front perspective view of a tourniquet system, showing a storage cavity and a tourniquet disposed outside of the storage cavity, according to one embodiment of the invention. There is illustrated, a base clothing layer 602 with a flap of flexible material 604 removably and fixedly coupled to the base clothing layer 602. The flap of flexible material includes a tab 606. When the flap of flexible material 604 is disposed over the base clothing layer 602 a storage cavity 608 is formed therebetween. A tourniquet 610 may be disposed within the storage cavity 608. The flap of flexible material 604 and the base clothing layer 602 may each have a plurality of snaps 634 for coupling together.

As shown, the base clothing layer 602 couples to the flap of flexible material 604 by a plurality of snaps 634. In one embodiment, the snaps 634 may be plastic snaps. However, the pull-release corrosion-proof coupling systems (see FIG. 5, items 518 and 526) may be any corrosion-proof coupling system for coupling the base clothing layer 602 to the flap of flexible material 604. For instance, the pull-release corrosion-proof coupling system may include hook and loop, plastic snaps, plastic buckles, buttons with eyelets and/or snaps, etc.

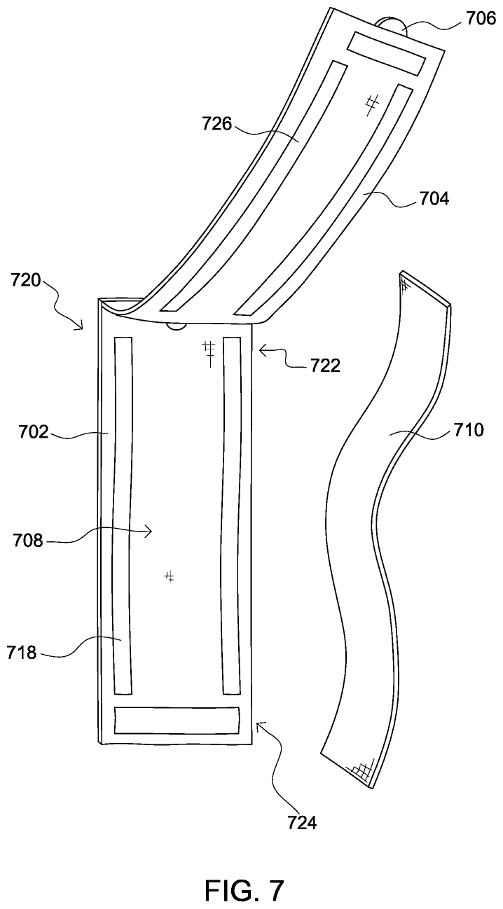

FIG. 7 illustrates a front perspective view of a tourniquet system, showing a storage cavity and a tourniquet disposed outside of the storage cavity, according to one embodiment of the invention. There is shown, a base layer 702 coupled to a flap of flexible material 704 with a tab 706. A storage cavity 708 is formed between the base layer 702 and the flap of flexible material 702. A tourniquet 710 may be stored within the storage cavity 708. The base layer 702 has a first pull-release corrosion-proof coupling system 718 that is disposed in a U-shape 720 with an open top end 722 and a closed bottom end 724. The first pull-release corrosion-proof coupling system 718 mates with a second pull-release corrosion-proof coupling system 726 that is disposed along the flap of flexible material 704.

The illustrated storage cavity 108 may be a pouch or pocket-like receptacle. In one embodiment, a tourniquet may be disposed within the storage cavity 108. The storage cavity 108 may be formed by fixedly or removably coupling the base clothing layer 102 and the flap of flexible material 104 to the shirt. The pouch may be coupled to the shirt by hook and loop, or the pouch may be sewn or stitched to the shirt. The pouch may be coupled to a different portion of the shirt, such as the trunk or body of the shirt. The pouch may also be coupled to another item or object, such as, but not limited to: a pair of pants; a jacket; a tool box; a surf board; and a first aid kit.

In operation of an embodiment, there may be a tourniquet system placed on a long sleeve shirt. The shirt may be a swim shirt, tactical unit shirt, first responder shirt, uniform shirt, and so on. The tourniquet system may be placed on both a left and a right sleeve of a shirt. The tourniquet system may include a storage cavity 708 with a loose, ready-to-use, folded-up tourniquet 710. The storage cavity 708 may be formed to the sleeve. For instance, the storage cavity 708 may be permanently formed to a sleeve by stitching, or the storage cavity 708 may be removably coupled to a sleeve by hook and loop. The storage cavity 708 may have a top and a bottom with four sides. The top of the storage cavity 708 may include a flap of flexible material 704 that may removably couple to a base layer 702 of the storage cavity 708 along three of the four sides. The flap of flexible material 704 may removably couple to the base layer by a pull-release corrosion-proof coupling system 718 and 726. The flap of flexible material 704 may be fixed to the base layer 702 along one side such as by stitching. The flap of flexible material 704 may be pulled open and away from the base layer 702 by grasping a tab 706 disposed at an end of the flap of flexible material 704, and pulling it upward and away, thereby detaching the pull-release corrosion-proof coupling system 718 and 726.

It is understood that the above-described embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

For example, it is envisioned that the tourniquet system may be placed anywhere along an article of clothing. For instance, the tourniquet system may be placed under an armpit, or on a forearm. Similarly, the tourniquet system may be a pocket that is on top of a shoulder or on the small of the back. Moreover, the tourniquet system may be a separate structure, such as a pouch, that is entirely removably coupled to a shirt. For example, the tourniquet system may be removably coupled to a garment by preparing a garment with a landing strip, such as of hook and loop, so that the entire tourniquet system may be coupled to the garment.

It is expected that there could be numerous variations of the design of this invention. It is envisioned that the flap of flexible material may open in a downward direction from shoulder to elbow, rather than in an upward direction from elbow to shoulder. More, a grommet or hole may be disposed at a different location along the pouch, and the pouch may have multiple grommets disposed along the pouch. It is also envisioned that the tab may have a different shape, such as triangular, rectangular, square, and so on. In addition, the tab may be an extension of the pouch so that the pouch is one continuous piece, or the tab may be a separate structure that is coupled to the pouch.

Finally, it is envisioned that the components of the device may be constructed of a variety of materials and textiles, such as but not limited to: plastic, spandex, nylon, metal, polyester, cotton, and/or rubber.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims. Further, it is contemplated that an embodiment may be limited to consist of or to consist essentially of one or more of the features, functions, structures, methods described herein.

What is claimed is:

1. An article of clothing configured to store a tourniquet, comprising:
a. a base clothing layer, including:
i. a first plurality of hook and loop attachment regions arranged in a U-shape, the U-shape having an open top end and a closed bottom end;
b. a flap of flexible material fixedly coupled to the base clothing layer adjacent to the top end of the U-shape, and extending downwardly coextensive with the first plurality of hook and loop attachment regions, and including:
i. a second plurality of hook and loop attachment regions positioned and configured to mate with the first plurality of hook and loop attachment regions; and
ii. a tab protruding beyond the second plurality of hook and loop attachment regions; and
c. wherein when the flap of flexible material is fully coupled to the base clothing layer by mating of the first plurality of hook and loop attachment regions and the second plurality of hook and loop attachment regions a storage cavity is formed.

2. The article of clothing of claim 1, further comprising a tourniquet disposed within the storage cavity.

3. The article of clothing of claim 2, wherein the tourniquet is not fixedly coupled to the base clothing layer.

4. The article of clothing of claim 1, wherein the flap of flexible material includes a hole disposed therethrough disposed near a top end of the U-shape.

5. The article of clothing of claim 1, wherein the article of clothing is a shirt and the base clothing layer is a sleeve of the shirt, and the top end of the U-shape is disposed closer to a shoulder of the shirt than the bottom end of the U-shape.

6. The article of clothing of claim 5, wherein the shirt is comprised of spandex and nylon.

7. The article of clothing of claim 5, wherein the shirt is comprised of spandex and polyester.

8. The article of clothing of claim 1, wherein the hook and loop attachment regions each include three patches:
a. a first patch positioned vertically along a side of the U-shape,
b. a second patch positioned horizontally along a base of the U-shape; and
c. a third patch positioned vertically along a side of the U-shape opposite the first patch.

9. An article of clothing configured to store a tourniquet, comprising:
a. a base clothing layer, including:
i. a first pull-release corrosion-proof coupling system arranged in a U-shape, the U-shape having an open top end and a closed bottom end,
b. a flap of flexible material fixedly coupled to the base clothing layer adjacent to the top end of the U-shape, and extending downwardly coextensive with the first pull-release corrosion-proof coupling system, and including:
i. a second pull-release corrosion-proof coupling system positioned and configured to mate with the first pull-release corrosion-proof coupling system; and
ii. a tab protruding beyond the second pull-release corrosion-proof coupling system; and
c. wherein when the flap of flexible material is fully coupled to the base clothing layer by mating of the first pull-release corrosion-proof coupling system and the second pull-release corrosion-proof coupling system a storage cavity is formed.

10. The article of clothing of claim 9, wherein the first pull-release corrosion-proof coupling system and the second pull-release corrosion-proof coupling system comprises a plurality of plastic snaps.

11. The article of clothing of claim 9, wherein the flap of flexible material is fixedly coupled toward a top of the article of clothing and releasably coupled toward a bottom of the article of clothing.

12. The article of clothing of claim 9, further comprising a tourniquet disposed within the storage cavity.

13. The article of clothing of claim 11, wherein the tourniquet is not fixedly coupled to the base clothing layer.

14. The article of clothing of claim 9, wherein the flap of flexible material includes a hole disposed therethrough disposed near a top end of the U-shape.

15. The article of clothing of claim 9, wherein the article of clothing is a shirt and the base clothing layer is a sleeve of the shirt, and the top end of the U-shape is disposed closer to a shoulder of the shirt than the bottom end of the U-shape.

16. The article of clothing of claim 15, wherein the shirt is comprised of spandex and nylon.

17. The article of clothing of claim 15, wherein the shirt is comprised of spandex and polyester.

18. The article of clothing of claim 9, wherein the pull-release corrosion-proof coupling systems each include three arrays:
a. a first array positioned vertically along a side of the U-shape,
b. a second array positioned horizontally along a base of the U-shape; and
c. a third array positioned vertically along a side of the U-shape opposite the first array.

19. An article of clothing configured to store a tourniquet, comprising:
a. a base layer removably coupleable to the article of clothing, including:
i. a first pull-release corrosion-proof coupling system, disposed along an inner surface of the base layer; and
b. a flap layer fixedly coupled to and disposed over the base layer opposite the article of clothing, wherein the flap layer is a flexible material including:
i. a second pull-release corrosion-proof coupling system positioned and configured to mate with the first pull-release corrosion-proof coupling system; and
ii. a tab protruding beyond the second pull-release corrosion-proof coupling system; and
iii. wherein when the flap layer is fully coupled to the base layer by mating of the first pull-release corrosion-proof coupling system and the second pull-release corrosion-proof coupling system a storage cavity is formed.

20. The article of clothing of claim 19, further comprising a tourniquet disposed within the storage cavity.
